(19) United States
(12) Patent Application Publication
(10) Pub. No.: US 2025/0131993 A1
(43) Pub. Date: Apr. 24, 2025

(54) APPARATUS AND METHODS FOR TRACKING PHARMACEUTICAL SAMPLES

(71) Applicant: Darren Peress, Tucson, AZ (US)

(72) Inventor: Darren Peress, Tucson, AZ (US)

(21) Appl. No.: 18/489,252

(22) Filed: Oct. 18, 2023

(51) Int. Cl. G16H 10/60; G16H 20/10

(52) U.S. Cl. CPC G16H 10/60; G16H 20/10

(57) ABSTRACT

The methods employ a camera and flexible text recognition algorithms implemented on a computing system or smart phone to scan printed or imprinted text on pharmaceutical sample boxes or bottles and determine therefrom the drug name, dose, lot number and expiration date. Operable communication with an inventory of available pharmaceutical samples enables updating the inventory to reflect dispensing samples to patients and receiving new samples. The systems can be connected (wirelessly or wired) to a practice's electronic medical record (EMR) database to enable up-to-date patient records.

### BACKGROUND

Pharmaceutical samples are regularly dispensed to patients by physicians or allied professionals in medical offices. Responsive to dispensing the pharmaceutical samples, the provider is expected to update the patient's medical record to include details of the transaction. However, labeling requirements for pharmaceutical samples (also referred to as medications) are less rigorous than for non-sample medicines, making it technically challenging for a provider to track the flow of pharmaceutical samples. Accordingly, improvements to tracking pharmaceutical samples are desirable.

### BRIEF DESCRIPTION OF THE DRAWINGS

FIG. 1 is a simplified technology environment in which embodiments of the present disclosure may be implemented.

FIG. 2 illustrates an example use case illustrating advantages provided by embodiments of the present disclosure.

FIGS. 3A, 3B, and 3C illustrate various displays generated by embodiments of the present disclosure.

FIG. 4 is an exemplary method as may be performed by embodiments of the present disclosure.

FIG. 5 is a block diagram of an example compute node that may include any of the embodiments disclosed herein.

FIG. 6 illustrates a multi-processor environment in which embodiments may be implemented.

FIG. 7 is a block diagram of an example processor unit to execute computer-executable instructions as part of implementing technologies described herein.

### DETAILED DESCRIPTION

Samples of many pharmaceutical samples or medications are regularly dispensed to patients by physicians or allied professionals in medical offices (hereinafter “provider(s)”). Responsive to dispensing the pharmaceutical samples, the provider is expected to update the patient's medical record to include the medication's name, dose, quantity, lot number, and expiration date. However, the standards for pharmaceutical labelling, which require a machine-readable label with a barcode that contains the medication name, dose, lot number, and expiration date, do not apply to pharmaceutical samples, presenting a technical challenge.

Some solutions involve having the provider manually update the patient's medical record in a print or electronic ledger. This can be cumbersome, as there may be multiple medications sampled to a patient, each of which require entering the medication name, dose, quantity, lot number and expiration date. Further, for a sampled medication, there may be more than one box/bottle, in which case the name, dose, quantity, lot number, and expiration date must be entered for every box/bottle dispensed. Manual entry of this information is cumbersome and prone to human error; consequently, many transactions are simply not tracked.

Not tracking the pharmaceutical samples can have a variety of adverse consequences, such as:
No record of who is prescribing pharmaceutical samples and who is receiving them;
Existing automated processes that normally alert a provider that the patient has a listed allergy to a pharmaceutical sample are not triggered because the transaction is not entered in the patient's medical record;
Patients cannot be contacted in the event of a medication recall;
Expired medications could be dispensed; and
There is no way to determine if pharmaceutical samples are diverted (from loss or theft)

Aspects of the present disclosure provide a technical solution to this technical problem and other related enhancements, in the form of apparatuses and methods for tracking pharmaceutical samples. The herein provided apparatuses and methods employ a camera and flexible text recognition algorithms to scan printed or imprinted text on pharmaceutical sample boxes or bottles and determine therefrom the drug name, dose, lot number and expiration date. Various embodiments can be connected (wirelessly or wired) to a practice's electronic medical record (EMR) database.

Embodiments can be detected with an observation of the use of a camera (such as a cellphone camera) to scan and track pharmaceutical samples. Embodiments can also be detected by inspecting the program modules implemented in a cellphone to identify the rules and program code to enable the tasks and operations described herein. A more detailed description of the aspects of the present disclosure follows a terminology section.

### Terminology

As used herein, the terms “processor unit”, “processing unit” or “processor” may refer to any device or portion of a device that processes electronic data from registers and/or memory to transform that electronic data into other electronic data that may be stored in registers and/or memory. A processor unit may be a system-on-a-chip (SOC), and/or include one or more digital signal processors (DSPs), application-specific integrated circuits (ASICs), central processing units (CPUs), graphics processing units (GPUs), general-purpose GPUs (GPGPUs), accelerated processing units (APUs), field-programmable gate arrays (FPGAs), neural network processing units (NPUs), data processor units (DPUs), accelerators (e.g., graphics accelerator, compression accelerator, artificial intelligence accelerator), controller cryptoprocessors (specialized processors that execute cryptographic algorithms within hardware), server processors, controllers, or any other suitable type of processor units. As such, the processor unit can be referred to as an XPU (or xPU).

As used herein, the term “module” refers to logic that may be implemented in a hardware component or device, software or firmware running on a processor unit, or a combination thereof, to perform one or more tasks or operations described in the present disclosure. Software and firmware may be embodied as instructions and/or data stored on non-transitory computer-readable storage media. As used herein, the term “circuitry” can comprise, singly or in any combination, non-programmable (hardwired) circuitry, programmable circuitry such as processor units, state machine circuitry, and/or firmware that stores instructions executable by programmable circuitry. Modules described herein may, collectively or individually, be embodied as circuitry that forms a part of a computing system. Thus, any of the modules can be implemented as circuitry, such as continuous itemset generation circuitry, entropy-based discretization circuitry. A computing system or processor referred to as being programmed to perform a method can be programmed to perform the method via software, hardware, firmware, or combinations thereof.

### Description of Aspects of the Present Disclosure

Example embodiments are hereinafter described in conjunction with the following drawing figures, wherein like numerals denote like elements. Figures are not necessarily to scale. As may be appreciated, certain terminology, such as “upper,”, “uppermost”, “lower,” “above,” “below,” “bottom,” and “top” refer to directions based on viewing the Figures to which reference is made. Further, terms such as “front,” “back,” “rear,”, “side”, “vertical”, and “horizontal” may describe an orientation and/or location within a consistent but arbitrary frame of reference which is made clear by reference to the text and the associated Figures describing the component under discussion. Such terminology may include the words specifically mentioned above, derivatives thereof, and words of similar import. For the sake of brevity, well-known structures and devices may be shown in block diagram form to facilitate a description thereof.

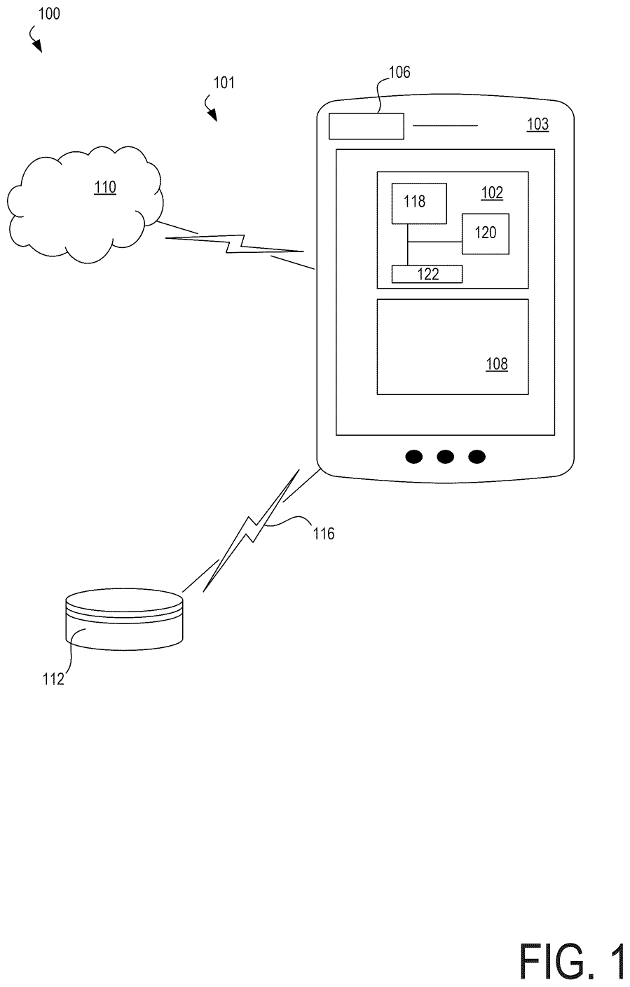

FIG. 1 is a simplified technology environment 100 in which a system 101 for tracking pharmaceutical samples may be implemented. The system 101 includes a computing system 103. The computing system 103 includes a program module 102, described in more detail below. The computing system 103 also includes a camera 106 system and a user interface 108 (also referred to as a human machine interface HMI). In environment 100, the computing system 103 has (wired or wireless) access to one or more storage components 112. The storage component(s) 112 may store a database of electronic medical records (EMRs) for patients that utilize a medical practice. The storage component 112 may also store a database of available pharmaceutical samples for a given medical practice. The computing system 103 may support both wired and wireless 116 communication protocols. In various embodiments, the computing system 103 may additionally be in operable communication with a network or cloud 110.

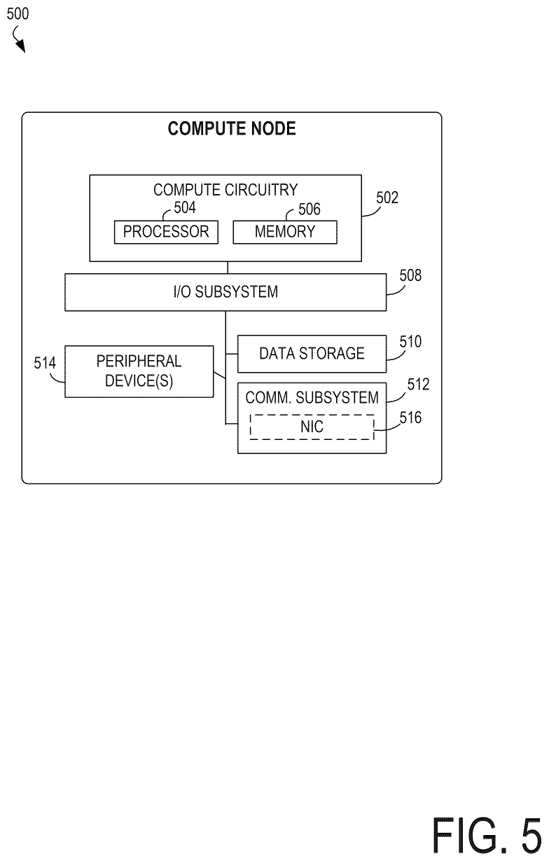

Various embodiments described herein implement the computing system 103 as a smart phone, therefore, examples below reference a smart phone 104. However, a “computing system” refers to any of a variety of computing devices and includes systems comprising multiple discrete physical components, as is referenced again in connection to FIGS. 5-7. For example, the computing system 103 can be any of a variety of computing systems, including mobile computing systems (e.g., smartphones, handheld computers, tablet computers, laptop computers, 2-in-1 convertible computers, portable all-in-one computers), and non-mobile computing systems (e.g., desktop computers, servers, workstations, or set-top boxes).

The camera 106 system includes the optical components, electronic components, and software as is generally available in a smart phone. The user interface 108 also includes the objects, electronic components, and software as is generally available in a smart phone (e.g., a display screen that may be touch sensitive, a keyboard, voice recognition, and the like). As may be appreciated, the smart phone 104 further includes the appropriate circuitry and program modules to provide communication with a computer, a network, or the cloud, as is generally expected by a smart phone; these modules are omitted to simplify the illustration.

The program module 102 integrates the rules and algorithms that cause the system 101 for tracking pharmaceutical samples to operate as described herein. The program module 102 may be implemented as software or as hardware. In an example, the program module 102 may be implemented as program code stored in memory 120, the program code, when executed by the processor 118, communicates through a communication fabric 122 to cause components of the system 101 to perform the tasks and operations attributed to it herein. In an alternative example, the program module 102 may be implemented as hardware, as a system on chip, as a multi-die assembly, as a chipset, or similar.

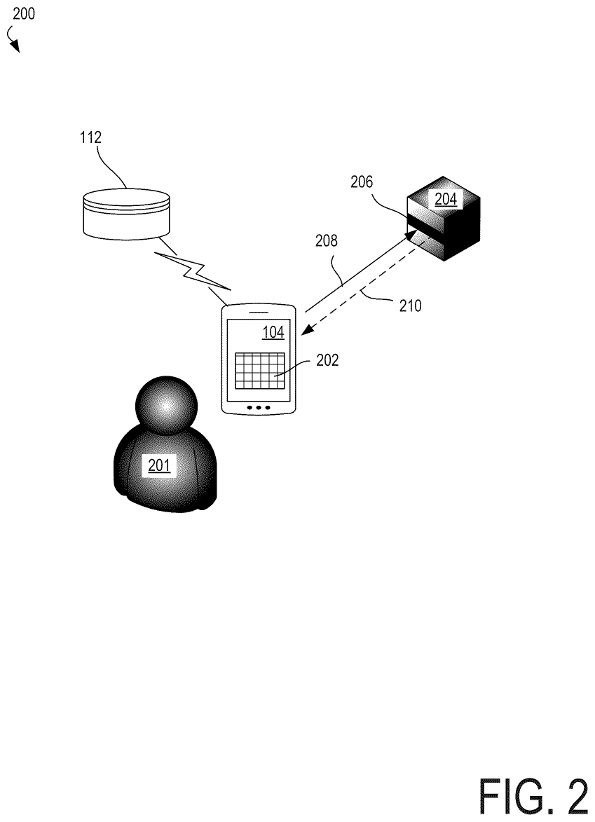

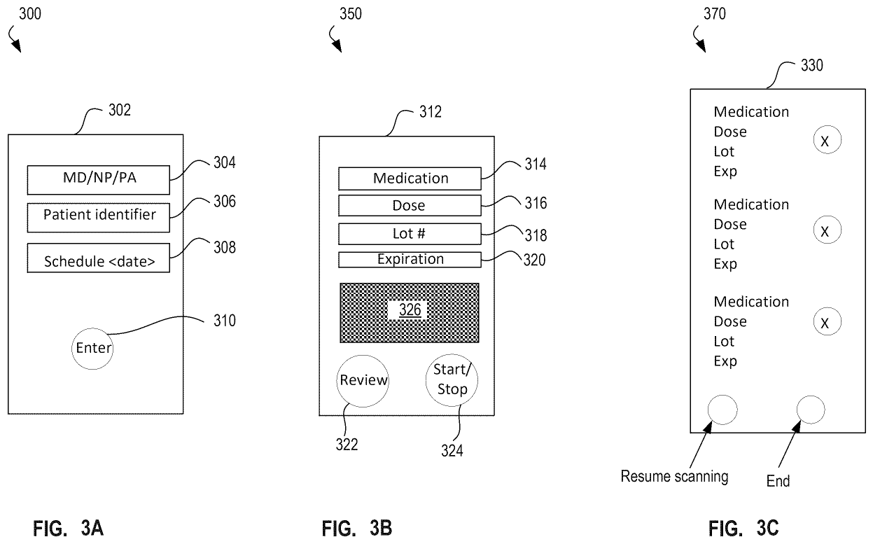

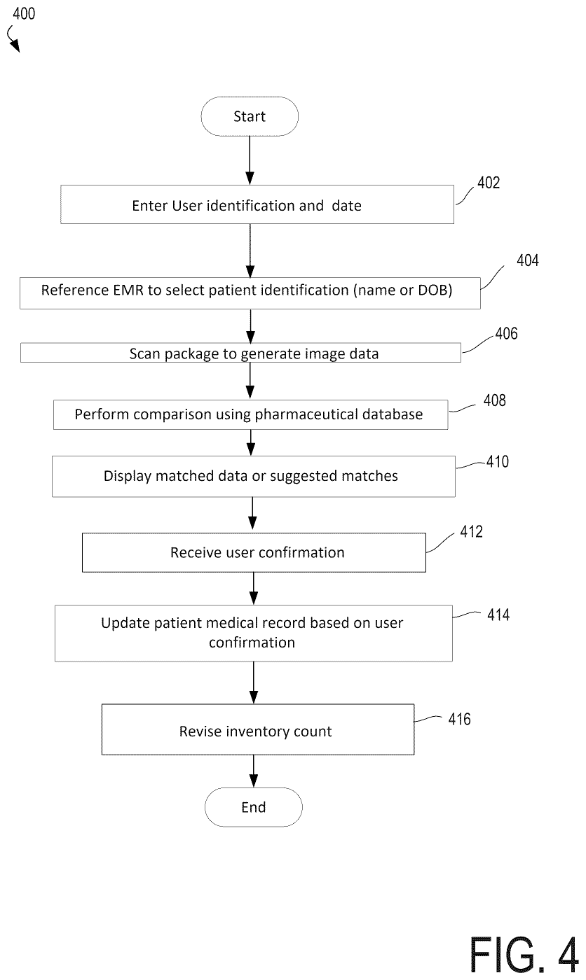

FIG. 2 illustrates an example use case. FIGS. 3A, 3B, and 3C are non-limiting examples of various displays generated on the smart phone 104 during operation of the system 101, and FIG. 4 illustrates an exemplary method for tracking pharmaceutical samples, in accordance with various embodiments. As mentioned above, in performance of the method 400, the tasks and operations associated with the system 101 may be distributed as a program product with predefined parameters, the program product can be stored in on-board memory 120 in a given computing system 103 and executed by the processor 118.

In the technology environment 200, a user 201 may interact with the system 101 for tracking pharmaceutical samples through a human machine interface (HMI) or user interface on the smart phone 104. The HMI includes a touch screen display panel 202 and may also include a microphone, a speaker, and may operate on voice commands, touch input, gesture control, or any combination thereof.

Various embodiments can be connected (wirelessly or wired) to a medical practice's record of available pharmaceutical samples and the medical practice's database of electronic medical records (EMRs) for its patients. These two databases may be externally located, as is generally indicated with the external storage component 112. In the generated display shown in embodiment 302, the user enters the user identification and date into tab 304 (at 402). Non-limiting examples of user identifications include a medical doctor, nurse practitioner, a medical assistant, or a physician's assistant. In other scenarios, the user may be a pharmaceutical sales representative. The system 101 can authenticate or authorize the user by referencing a pre-configured approved list, or if an invalid user attempts to use the system 101, the system 101 can prevent the user from further interaction.

The user enters a patient identification into tab 306; the patient identification may be a name, a date of birth, or the like. At 404, the system 101 references the practice's storage component 112 using the patient identification to find the patient's EMR. In FIG. 4, at 404, the system 101 receives the patient identification, e.g., via the touch screen display, and references the database in storage component 112 to obtain an EMR for the patient responsive thereto. In response, the system 101 may display the patient's name and patient's practice physician, or similar, for the user to view and confirm. The user confirms the submission, e.g., using the enter button 310.

After confirming the intended patient's name, the user can enter a scan request (for example, by touching a prompt) to cause the camera 106 system to scan (at 406) a package 204 containing a pharmaceutical sample. The package 204 includes a print/imprint 206 of which the camera 106 system will create image data therefrom. Arrow 208 represents the camera focus aimed at the print/imprint on the package, and arrow 210 represents the feedback light to the camera 106 system therefrom.

The program module 102 receives image data from the camera 106 system, and responsive thereto, references the pre-loaded database of pharmaceutical data comprising medication names, doses, lot numbers and expiration dates, to perform a comparison (at 408) operation to search for matching data (also referred to as a match, and “matched data”). Performing the comparison at 408 may include employing a text recognition and/or object recognition operation. In some embodiments, a minimum of the lot number (tab 318) is required to begin the comparison operation.

At 410, the program module 102 displays the matched data, as illustrated in display 312 of embodiment 350. The data associated with the match (“matched data”) will include one or more of a medication name (tab 314), dose (tab 316), lot number (tab 318), and expiration date (tab 320). In various embodiments, the system 101 will also display a camera image 326 for review by the user.

On occasion, some of the text printed or imprinted may be difficult to match, causing a match fault, meaning that no match is found. In an example, a match fault may occur when the comparison operation misreads text or determines there is missing text between the image data and the pharmaceutical sample data in the pre-loaded database. A common reason for misreading text is that some characters can look very similar; non-limiting examples of this include a G and a 6, or a B and an 8. Responsive to a match fault, embodiments may perform an approximate string-matching algorithm in these situations, to thereby suggest potential matches. Alternatively, embodiments may adjust filters on the camera to enhance the image and return to performing the comparison operation.

Upon completion of the algorithms described above, the system 101 enables (prompts) the user/provider to review the generated information and to manually edit the medical data or correct characters as required for accuracy. Accordingly, additional display options include a review button 322. Selecting the review button can open up display 330, for the user to individually confirm or reject displayed matched data (e.g., responsive to an incorrect match), and a start/stop button 324. As illustrated in embodiment 370, a user can delete individual suggested matched data, resume scanning, or end the scan and match process with the touch of a button.

The user may confirm or select the matched data at 412. Embodiments can search the patient's medical record and generate an alert when the medical record indicates that there is a reported allergy to the medication name in the matched data. Embodiments may update the patient's medical record based on the user confirmation at 414 to dispense the sample to the patient. The updated medical record can be saved in a cloud-based database.

Embodiments may also keep a log of transactions and a log of inventory of available pharmaceutical samples. Upon completion of the transaction, the system 101 will revise the inventory count in accordance with the transaction. Revising the inventory count includes decrementing the inventory when samples are dispensed to patients and incrementing the inventory count when new samples are received from pharmaceutical sales representatives.

Various embodiments may reference the log of transactions to identify patient's names that are associated with pharmaceutical samples that have been recalled so that they may notify those patients about the recall. In various embodiments, authenticated user identification can be used by the system 101 to lock and unlock storage closets that contain the pharmaceutical samples.

Thus, architectures and methods for tracking pharmaceutical samples have been provided. While embodiments above depict the computing system as a smart phone 104, it is understood by those with skill in the art that the rules and algorithms of the present disclosure may be implemented in a variety of other compute environments. The following description illustrates various context for usage and application of provided aspects of the present disclosure.

In the simplified example depicted in FIG. 5, a compute node 500 includes a compute engine (referred to herein as “compute circuitry”) 502, an input/output (I/O) subsystem 508, data storage 510, a communication circuitry subsystem 512, and, optionally, one or more peripheral devices 514. With respect to the present example, the compute node 500 or compute circuitry 502 may perform the operations and tasks attributed to the system 101. In other examples, respective compute nodes 500 may include other or additional components, such as those typically found in a computer (e.g., a display, peripheral devices, etc.). Additionally, in some examples, one or more of the illustrative components may be incorporated in, or otherwise form a portion of, another component.

In some examples, the compute node 500 may be embodied as a single device such as an integrated circuit, an embedded system, a field-programmable gate array (FPGA), a system-on-a-chip (SOC), or other integrated system or device. In the illustrative example, the compute node 500 includes or is embodied as a processor 504 and a memory 506. The processor 504 may be embodied as any type of processor capable of performing the functions described herein (e.g., executing compile functions and executing an application). For example, the processor 504 may be embodied as a multi-core processor(s), a microcontroller, a processing unit, a specialized or special purpose processing unit, or other processor or processing/controlling circuit.

In some examples, the processor 504 may be embodied as, include, or be coupled to an FPGA, an application specific integrated circuit (ASIC), reconfigurable hardware or hardware circuitry, or other specialized hardware to facilitate performance of the functions described herein. Also in some examples, the processor 504 may be embodied as a specialized x-processing unit (xPU) also known as a data processing unit (DPU), infrastructure processing unit (IPU), or network processing unit (NPU). Such an xPU may be embodied as a standalone circuit or circuit package, integrated within an SOC, or integrated with networking circuitry (e.g., in a SmartNIC, or enhanced SmartNIC), acceleration circuitry, storage devices, or AI hardware (e.g., GPUs or programmed FPGAs). Such an xPU may be designed to receive programming to process one or more data streams and perform specific tasks and actions for the data streams (such as hosting microservices, performing service management or orchestration, organizing, or managing server or data center hardware, managing service meshes, or collecting and distributing telemetry), outside of the CPU or general-purpose processing hardware. However, it will be understood that a xPU, a SOC, a CPU, and other variations of the processor 504 may work in coordination with each other to execute many types of operations and instructions within and on behalf of the compute node 500.

The memory 506 may be embodied as any type of volatile (e.g., dynamic random-access memory (DRAM), etc.) or non-volatile memory or data storage capable of performing the functions described herein. Volatile memory may be a storage medium that requires power to maintain the state of data stored by the medium. Non-limiting examples of volatile memory may include various types of random-access memory (RAM), such as DRAM or static random-access memory (SRAM). One particular type of DRAM that may be used in a memory module is synchronous dynamic random-access memory (SDRAM).

In an example, the memory device is a block addressable memory device, such as those based on NAND or NOR technologies. A memory device may also include a three-dimensional crosspoint memory device (e.g., Intel® 3D XPoint™ memory), or other byte addressable write-in-place nonvolatile memory devices. The memory device may refer to the die itself and/or to a packaged memory product. In some examples, 3D crosspoint memory (e.g., Intel® 3D XPoint™ memory) may comprise a transistor-less stackable cross point architecture in which memory cells sit at the intersection of word lines and bit lines and are individually addressable and in which bit storage is based on a change in bulk resistance. In some examples, all or a portion of the memory 506 may be integrated into the processor 504. The memory 506 may store various software and data used during operation such as one or more applications, data operated on by the application(s), libraries, and drivers.

The compute circuitry 502 is communicatively coupled to other components of the compute node 500 via the I/O subsystem 508, which may be embodied as circuitry and/or components to facilitate input/output operations with the compute circuitry 502 (e.g., with the processor 504 and/or the main memory 506) and other components of the compute circuitry 502. For example, the I/O subsystem 508 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some examples, the I/O subsystem 508 may form a portion of a system-on-a-chip (SoC) and be incorporated, along with one or more of the processor 504, the memory 506, and other components of the compute circuitry 502, into the compute circuitry 502.

The one or more illustrative data storage devices 510 may be embodied as any type of devices configured for short-term or long-term storage of data such as, for example, memory devices and circuits, memory cards, hard disk drives, solid-state drives, or other data storage devices. Individual data storage devices 510 may include a system partition that stores data and firmware code for the data storage device 510. Individual data storage devices 510 may also include one or more operating system partitions that store data files and executables for operating systems depending on, for example, the type of compute node 500.

The communication subsystem 512 may be embodied as any communication circuit, device, transceiver circuit, or collection thereof, capable of enabling communications over a network between the compute circuitry 502 and another compute device (e.g., an edge gateway of an implementing edge computing system).

The communication subsystem 512 may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). IEEE 802.16 compatible Broadband Wireless Access (BWA) networks are generally referred to as WiMAX networks, an acronym that stands for Worldwide Interoperability for Microwave Access, which is a certification mark for products that pass conformity and interoperability tests for the IEEE 802.16 standards. The communication subsystem 512 may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication subsystem 512 may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication subsystem 512 may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. The communication subsystem 512 may operate in accordance with other wireless protocols in other embodiments. The communication subsystem 512 may include an antenna to facilitate wireless communications and/or to receive other wireless communications (such as AM or FM radio transmissions).

In some embodiments, the communication subsystem 512 may manage wired communications, such as electrical, optical, or any other suitable communication protocols (e.g., IEEE 802.3 Ethernet standards). As noted above, the communication subsystem 512 may include multiple communication components. For instance, a first communication subsystem 512 may be dedicated to shorter-range wireless communications such as Wi-Fi or Bluetooth, and a second communication subsystem 512 may be dedicated to longer-range wireless communications such as global positioning system (GPS), EDGE, GPRS, CDMA, WiMAX, LTE, EV-DO, or others. In some embodiments, a first communication subsystem 512 may be dedicated to wireless communications, and a second communication subsystem 512 may be dedicated to wired communications.

The illustrative communication subsystem 512 includes an optional network interface controller (NIC) 520, which may also be referred to as a host fabric interface (HFI). The NIC 520 may be embodied as one or more add-in-boards, daughter cards, network interface cards, controller chips, chipsets, or other devices that may be used by the compute node 500 to connect with another compute device (e.g., an edge gateway node). In some examples, the NIC 520 may be embodied as part of a system-on-a-chip (SoC) that includes one or more processors or included on a multichip package that also contains one or more processors. In some examples, the NIC 520 may include a local processor (not shown) and/or a local memory (not shown) that are both local to the NIC 520. In such examples, the local processor of the NIC 520 may be capable of performing one or more of the functions of the compute circuitry 502 described herein. Additionally, or alternatively, in such examples, the local memory of the NIC 520 may be integrated into one or more components of the client compute node at the board level, socket level, chip level, and/or other levels.

Additionally, in some examples, a respective compute node 500 may include one or more peripheral devices 514. Such peripheral devices 514 may include any type of peripheral device found in a compute device or server such as audio input devices, a display, other input/output devices, interface devices, and/or other peripheral devices, depending on the particular type of the compute node 500. In further examples, the compute node 500 may be embodied by a respective edge compute node (whether a client, gateway, or aggregation node) in an edge computing system or like forms of appliances, computers, subsystems, circuitry, or other components.

In other examples, the compute node 500 may be embodied as any type of device or collection of devices capable of performing various compute functions. Respective compute nodes 500 may be embodied as a type of device, appliance, computer, or other “thing” capable of communicating with other compute nodes that may be edge, networking, or endpoint components. For example, a compute device may be embodied as a personal computer, server, smartphone, a mobile compute device, a smart appliance, smart camera, an in-vehicle compute system (e.g., a navigation system), a weatherproof or weather-sealed computing appliance, a self-contained device within an outer case, shell, etc., or other device or system capable of performing the described functions.

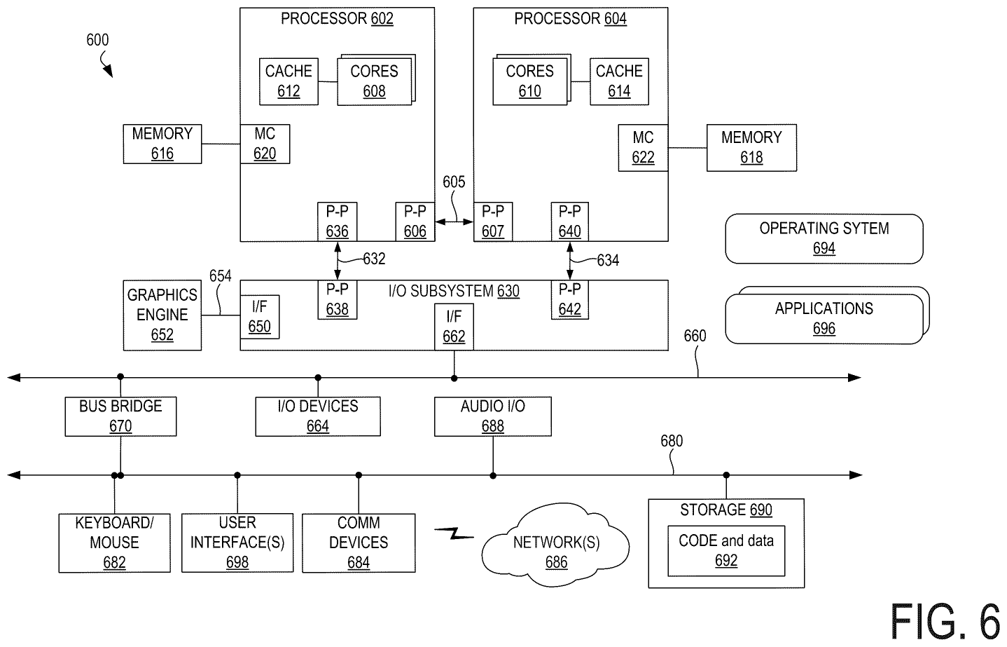

FIG. 6 illustrates a multi-processor environment in which embodiments may be implemented. The simplified processor illustration in program module 102 can be implemented as one or more of processor units 602 and 604. Processor units 602 and 604 further comprise cache memories 612 and 614, respectively. The cache memories 612 and 614 can store data (e.g., instructions) utilized by one or more components of the processor units 602 and 604, such as the processor cores 608 and 610. The cache memories 612 and 614 can be part of a memory hierarchy for the computing system 600. For example, the cache memories 612 can locally store data that is also stored in a memory 616 to allow for faster access to the data by the processor unit 602. In some embodiments, the cache memories 612 and 614 can comprise multiple cache levels, such as level 1 (L1), level 2 (L2), level 3 (L3), level 4 (L4) and/or other caches or cache levels. In some embodiments, one or more levels of cache memory (e.g., L2, L3, L4) can be shared among multiple cores in a processor unit or among multiple processor units in an integrated circuit component. In some embodiments, the last level of cache memory on an integrated circuit component can be referred to as a last level cache (LLC). One or more of the higher levels of cache levels (the smaller and faster caches) in the memory hierarchy can be located on the same integrated circuit die as a processor core and one or more of the lower cache levels (the larger and slower caches) can be located on an integrated circuit dies that are physically separate from the processor core integrated circuit dies.

Although the computing system 600 is shown with two processor units, the computing system 600 can comprise any number of processor units. Further, a processor unit can comprise any number of processor cores. A processor unit can take various forms such as a central processing unit (CPU), a graphics processing unit (GPU), general-purpose GPU (GPGPU), accelerated processing unit (APU), field-programmable gate array (FPGA), neural network processing unit (NPU), data processor unit (DPU), accelerator (e.g., graphics accelerator, digital signal processor (DSP), compression accelerator, artificial intelligence (AI) accelerator), controller, or other types of processing units. As such, the processor unit can be referred to as an XPU (or xPU). Further, a processor unit can comprise one or more of these various types of processing units. In some embodiments, the computing system comprises one processor unit with multiple cores, and in other embodiments, the computing system comprises a single processor unit with a single core. As used herein, the terms “processor unit” and “processing unit” can refer to any processor, processor core, component, module, engine, circuitry, or any other processing element described or referenced herein.

In some embodiments, the computing system 600 can comprise one or more processor units that are heterogeneous or asymmetric to another processor unit in the computing system. There can be a variety of differences between the processing units in a system in terms of a spectrum of metrics of merit including architectural, microarchitectural, thermal, power consumption characteristics, and the like. These differences can effectively manifest themselves as asymmetry and heterogeneity among the processor units in a system.

The processor units 602 and 604 can be located in a single integrated circuit component (such as a multi-chip package (MCP) or multi-chip module (MCM)) or they can be located in separate integrated circuit components. An integrated circuit component comprising one or more processor units can comprise additional components, such as embedded DRAM, stacked high bandwidth memory (HBM), shared cache memories (e.g., L3, L4, LLC), input/output (I/O) controllers, or memory controllers. Any of the additional components can be located on the same integrated circuit die as a processor unit, or on one or more integrated circuit dies separate from the integrated circuit dies comprising the processor units. In some embodiments, these separate integrated circuit dies can be referred to as “chiplets”. In some embodiments where there is heterogeneity or asymmetry among processor units in a computing system, the heterogeneity or asymmetric can be among processor units located in the same integrated circuit component. In embodiments where an integrated circuit component comprises multiple integrated circuit dies, interconnections between dies can be provided by the package substrate, one or more silicon interposers, one or more silicon bridges embedded in the package substrate (such as Intel® embedded multi-die interconnect bridges (EMIBs)), or combinations thereof.

Processor units 602 and 604 further comprise memory controller logic (MC) 620 and 622. As shown in FIG. 6, MCs 620 and 622 control memories 616 and 618 coupled to the processor units 602 and 604, respectively. The memories 616 and 618 can comprise various types of volatile memory (e.g., dynamic random-access memory (DRAM), static random-access memory (SRAM)) and/or non-volatile memory (e.g., flash memory, chalcogenide-based phase-change non-volatile memories), and comprise one or more layers of the memory hierarchy of the computing system. While MCs 620 and 622 are illustrated as being integrated into the processor units 602 and 604, in alternative embodiments, the MCs can be external to a processor unit.

Processor units 602 and 604 are coupled to an Input/Output (I/O) subsystem 630 via point-to-point interconnections 632 and 634. The point-to-point interconnection 632 connects a point-to-point interface 636 of the processor unit 602 with a point-to-point interface 638 of the I/O subsystem 630, and the point-to-point interconnection 634 connects a point-to-point interface 640 of the processor unit 604 with a point-to-point interface 642 of the I/O subsystem 630. Input/Output subsystem 630 further includes an interface 650 to couple the I/O subsystem 630 to a graphics engine 652. The I/O subsystem 630 and the graphics engine 652 are coupled via a bus 654.

The Input/Output subsystem 630 is further coupled to a first bus 660 via an interface 662. The first bus 660 can be a Peripheral Component Interconnect Express (PCIe) bus or any other type of bus. Various I/O devices 664 can be coupled to the first bus 660. A bus bridge 670 can couple the first bus 660 to a second bus 680. In some embodiments, the second bus 680 can be a low pin count (LPC) bus. Various devices can be coupled to the second bus 680 including, for example, a keyboard/mouse 682, audio I/O devices 688, and a storage device 690, such as a hard disk drive, solid-state drive, or another storage device for storing computer-executable instructions (code) 692 or data. The code 692 can comprise computer-executable instructions for performing methods described herein. Additional components that can be coupled to the second bus 680 include communication device(s) 684, which can provide for communication between the computing system 600 and one or more wired or wireless networks 686 (e.g. Wi-Fi, cellular, or satellite networks) via one or more wired or wireless communication links (e.g., wire, cable, Ethernet connection, radio-frequency (RF) channel, infrared channel, Wi-Fi channel) using one or more communication standards (e.g., IEEE 802.11 standard and its supplements).

In embodiments where the communication devices 684 support wireless communication, the communication devices 684 can comprise wireless communication components coupled to one or more antennas to support communication between the computing system 600 and external devices. The wireless communication components can support various wireless communication protocols and technologies such as Near Field Communication (NFC), IEEE 802.11 (Wi-Fi) variants, WiMax, Bluetooth, Zigbee, 4G Long Term Evolution (LTE), Code Division Multiplexing Access (CDMA), Universal Mobile Telecommunication System (UMTS) and Global System for Mobile Telecommunication (GSM), and 5G broadband cellular technologies. In addition, the wireless modems can support communication with one or more cellular networks for data and voice communications within a single cellular network, between cellular networks, or between the computing system and a public switched telephone network (PSTN).

The system 600 can comprise removable memory such as flash memory cards (e.g., SD (Secure Digital) cards), memory sticks, Subscriber Identity Module (SIM) cards). The memory in system 600 (including caches 612 and 614, memories 616 and 618, and storage device 690) can store data and/or computer-executable instructions for executing an operating system 694 and application programs 696. Example data includes web pages, text messages, images, sound files, and video data biometric thresholds for particular users or other data sets to be sent to and/or received from one or more network servers or other devices by the system 600 via the one or more wired or wireless networks 686, or for use by the system 600. The system 600 can also have access to external memory or storage (not shown) such as external hard drives or cloud-based storage.

The operating system 694 (also simplified to “OS” herein) can control the allocation and usage of the components illustrated in FIG. 6 and support the one or more application programs 696. The application programs 696 can include common computing system applications (e.g., email applications, calendars, contact managers, web browsers, messaging applications) as well as other computing applications.

The computing system 600 can support various additional input devices, represented generally as user interfaces 698, such as a touchscreen, microphone, monoscopic camera, stereoscopic camera, trackball, touchpad, trackpad, proximity sensor, light sensor, electrocardiogram (ECG) sensor, PPG (photoplethysmogram) sensor, galvanic skin response sensor, and one or more output devices, such as one or more speakers or displays. Other possible input and output devices include piezoelectric and other haptic I/O devices. Any of the input or output devices can be internal to, external to, or removably attachable with the system 600. External input and output devices can communicate with the system 600 via wired or wireless connections.

In addition, one or more of the user interfaces 698 may be natural user interfaces (NUIs). For example, the operating system 694 or applications 696 can comprise speech recognition logic as part of a voice user interface that allows a user to operate the system 600 via voice commands. Further, the computing system 600 can comprise input devices and logic that allows a user to interact with computing the system 600 via body, hand or face gestures. For example, a user's hand gestures can be detected and interpreted to provide input to a gaming application.

The I/O devices 664 can include at least one input/output port comprising physical connectors (e.g., USB, IEEE 1394 (FireWire), Ethernet, RS-232), a power supply (e.g., battery), a global satellite navigation system (GNSS) receiver (e.g., GPS receiver); a gyroscope; an accelerometer; and/or a compass. A GNSS receiver can be coupled to a GNSS antenna. The computing system 600 can further comprise one or more additional antennas coupled to one or more additional receivers, transmitters, and/or transceivers to enable additional functions.

[interconnections] In addition to those already discussed, integrated circuit components, integrated circuit constituent components, and other components in the computing system 694 can communicate with interconnect technologies such as Intel@QuickPath Interconnect (QPI), Intel@Ultra Path Interconnect (UPI), Computer Express Link (CXL), cache coherent interconnect for accelerators (CCIX®), serializer/deserializer (SERDES), Nvidia® NVLink, ARM Infinity Link, Gen-Z, or Open Coherent Accelerator Processor Interface (OpenCAPI). Other interconnect technologies may be used and a computing system 694 may utilize more or more interconnect technologies.

It is to be understood that FIG. 6 illustrates only one example computing system architecture. Computing systems based on alternative architectures can be used to implement technologies described herein. For example, instead of the processors 602 and 604 and the graphics engine 652 being located on discrete integrated circuits, a computing system can comprise an SoC (system-on-a-chip) integrated circuit incorporating multiple processors, a graphics engine, and additional components. Further, a computing system can connect its constituent component via bus or point-to-point configurations different from that shown in FIG. 6. Moreover, the illustrated components in FIG. 6 are not required or all-inclusive, as shown components can be removed and other components added in alternative embodiments.

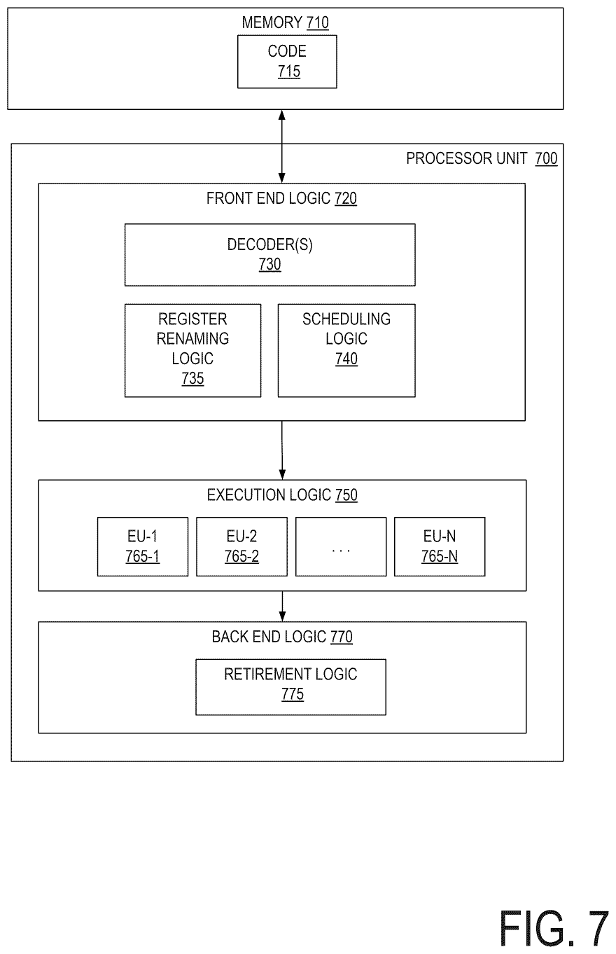

FIG. 7 is a block diagram of an example processor unit 700 to execute computer-executable instructions as part of implementing technologies described herein. The processor unit 700 can be a single-threaded core or a multithreaded core in that it may include more than one hardware thread context (or “logical processor”) per processor unit.

FIG. 7 also illustrates a memory 710 coupled to the processor unit 700. The memory 710 can be any memory described herein or any other memory known to those of skill in the art. The memory 710 can store computer-executable instructions 715 (code) executable by the processor unit 700.

The processor unit comprises front-end logic 720 that receives instructions from the memory 710. An instruction can be processed by one or more decoders 730. The decoder 730 can generate as its output a micro-operation such as a fixed width micro-operation in a predefined format, or generate other instructions, microinstructions, or control signals, which reflect the original code instruction. The front-end logic 720 further comprises register renaming logic 735 and scheduling logic 740, which generally allocate resources and queues operations corresponding to converting an instruction for execution.

The processor unit 700 further comprises execution logic 750, which comprises one or more execution units (EUs) 765-1 through 765-N. Some processor unit embodiments can include a few execution units dedicated to specific functions or sets of functions. Other embodiments can include only one execution unit or one execution unit that can perform a particular function. The execution logic 750 performs the operations specified by code instructions. After completion of execution of the operations specified by the code instructions, back-end logic 770 retires instructions using retirement logic 775. In some embodiments, the processor unit 700 allows out of order execution but requires in-order retirement of instructions. Retirement logic 775 can take a variety of forms as known to those of skill in the art (e.g., re-order buffers or the like).

The processor unit 700 is transformed during execution of instructions, at least in terms of the output generated by the decoder 730, hardware registers and tables utilized by the register renaming logic 735, and any registers (not shown) modified by the execution logic 750.

While at least one embodiment has been presented in the foregoing detailed description, it should be appreciated that a vast number of variations exist. It should also be appreciated that the disclosed embodiments are only examples, and are not intended to limit the scope, applicability, or configuration of the disclosure in any way. Rather, the foregoing detailed description will provide those skilled in the art with a convenient road map for implementing the disclosed aspects of the present disclosure. Various changes can be made in the function and arrangement of elements without departing from the scope of the disclosure as set forth in the appended claims and the legal equivalents thereof.

Any of the disclosed methods (or a portion thereof) can be implemented as computer-executable instructions or a computer program product. Such instructions can cause a computing system or one or more processor units capable of executing computer-executable instructions to perform any of the disclosed methods. As used herein, the term “computer” refers to any computing system, device, or machine described or mentioned herein as well as any other computing system, device, or machine capable of executing instructions. Thus, the term “computer-executable instruction” refers to instructions that can be executed by any computing system, device, or machine described or mentioned herein as well as any other computing system, device, or machine capable of executing instructions.

The computer-executable instructions or computer program products as well as any data created and/or used during implementation of the disclosed technologies can be stored on one or more tangible or non-transitory computer-readable storage media, such as volatile memory (e.g., DRAM, SRAM), non-volatile memory (e.g., flash memory, chalcogenide-based phase-change non-volatile memory) optical media discs (e.g., DVDs, CDs), and magnetic storage (e.g., magnetic tape storage, hard disk drives). Computer-readable storage media can be contained in computer-readable storage devices such as solid-state drives, USB flash drives, and memory modules. Alternatively, any of the methods disclosed herein (or a portion) thereof may be performed by hardware components comprising non-programmable circuitry. In some embodiments, any of the methods herein can be performed by a combination of non-programmable hardware components and one or more processing units executing computer-executable instructions stored on computer-readable storage media.

The computer-executable instructions can be part of, for example, an operating system of the computing system, an application stored locally to the computing system, or a remote application accessible to the computing system (e.g., via a web browser). Any of the methods described herein can be performed by computer-executable instructions performed by a single computing system or by one or more networked computing systems operating in a network environment. Computer-executable instructions and updates to the computer-executable instructions can be downloaded to a computing system from a remote server.

Further, it is to be understood that implementation of the disclosed technologies is not limited to any specific computer language or program. For instance, the disclosed technologies can be implemented by software written in C++, C#, Java, Perl, Python, JavaScript, Adobe Flash, C#, assembly language, or any other programming language. Likewise, the disclosed technologies are not limited to any particular computer system or type of hardware.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, cable (including fiber optic cable), magnetic communications, electromagnetic communications (including RF, microwave, ultrasonic, and infrared communications), electronic communications, or other such communication means.

Additionally, theories of operation, scientific principles, or other theoretical descriptions presented herein in reference to the apparatuses or methods of this disclosure have been provided for the purposes of better understanding and are not intended to be limiting in scope. The apparatuses and methods in the appended claims are not limited to those apparatuses and methods that function in the manner described by such theories of operation.

As used herein, phrases such as “embodiments,” “an aspect of the present disclosure,” “various aspects of the present disclosure,” “some aspects of the present disclosure,” and the like, indicate that some aspects of the present disclosure may have some, all, or none of the features described for other aspects of the present disclosure. “First,” “second,” “third,” and the like describe a common object and indicate different instances of like objects being referred to; unless specifically stated, they do not imply a given sequence, either temporally or spatially, in ranking, or any other manner. In accordance with patent application parlance, “connected” indicates elements that are in direct physical or electrical contact with each other and “coupled” indicates elements that co-operate or interact with each other, coupled elements may or may not be in direct physical or electrical contact. Furthermore, the terms “comprising,” “including,” “having,” and the like, are utilized synonymously to denote non-exclusive inclusions.

As used in this application and the claims, a list of items joined by the term “at least one of” can mean any combination of the listed terms. For example, the phrase “at least one of A, B and C” can mean A; B; C; A and B; A and C; B and C; or A, B, and C. Similarly, as used in this application and the claims, a list of items joined by the term “one or more of” can mean any combination of the listed terms. For example, the phrase “one or more of A, B and C” can mean A; B; C; A and B; A and C; B and C; or A, B, and C.

What is claimed is:

1. An apparatus comprising:
a camera system configured to create image data;
a touch screen display;
a database comprising an inventory of pharmaceutical samples;
a processor operably connected to the touch screen display, the database, and the camera system; and
instructions executable by the processor to:
receive a scan request via the touch screen display;
cause the camera system to create the image data responsive to the scan request;
compare the image data to the inventory of pharmaceutical samples;
display matched data on the touch screen display, responsive to finding matched data; and
receive a user input confirming the matched data.

2. The apparatus of claim 1, wherein the instructions further cause the processor to perform an approximate string-matching algorithm and suggest a potential matched data based thereon, responsive to not finding matched data.

3. The apparatus of claim 2, wherein the instructions further cause the processor to receive a user input confirming or rejecting the potential matched data.

4. The apparatus of claim 1, wherein the instructions further cause the processor to adjust the camera system to enhance the image data, responsive to not finding matched data.

5. The apparatus of claim 1, wherein the database further comprises electronic medical records (EMRs).

6. The apparatus of claim 5, wherein the instructions further cause the processor to receive a patient identification via the touch screen display and reference the database to obtain an EMR for the patient responsive thereto.

7. The apparatus of claim 6 wherein the instructions are further to:
search the EMR for the patient for an indication of an allergy to the matched data; and
display an alert to the allergy responsive to the indication of the allergy.

8. The apparatus of claim 6, wherein the instructions further cause the processor to:
receive a user input to update the EMR for the patient; and
update the EMR for the patient responsive thereto.

9. The apparatus of claim 8, wherein the instructions are further to update the inventory of pharmaceutical samples responsive to the update to the EMR for the patient.

10. The apparatus of claim 8 wherein the instructions are further to save the matched data and at least some of the EMR for the patient to a record in a cloud.

11. A system, comprising:
a storage component comprising an inventory of pharmaceutical samples and a plurality of electronic medical records (EMRs); and
a smart phone with a camera system, the smart phone in operable communication with the storage component;
wherein the smart phone comprises a program module programmed to:
display a prompt on the smart phone for a user identification, a date, and a patient identification;
authenticate a user based on the user identification;
reference the EMRs to find an EMR for a patient associated with the patient identification;
cause the camera system to scan a package and generate image data therefrom responsive to a user scan request;
perform a compare operation using the image data and the inventory of pharmaceutical samples;
display matched data; and
receive a user input confirming the matched data and the patient.

12. The system of claim 11, wherein the program module is further programmed to perform an approximate string-matching algorithm and display a potential matched data based thereon, responsive to not finding matched data.

13. The system of claim 12, wherein the program module is further programmed to receive a user input confirming or rejecting the potential matched data.

14. The system of claim 11, wherein the program module is further programmed to adjust the camera system to enhance the image data, responsive to not finding matched data.

15. The system of claim 11, wherein the program module is further programmed to:
search the EMR for the patient for an indication of an allergy to the matched data; and
display an alert to the allergy responsive to the indication of the allergy.

16. The system of claim 11, wherein the program module is further programmed to:
receive a user input to update the EMR for the patient; and
update the EMR for the patient responsive thereto.

17. The system of claim 16, wherein the program module is further programmed to update the inventory of pharmaceutical samples responsive to the update to the EMR for the patient.

18. The system of claim 11, wherein the program module is further programmed to save the matched data and at least some of the EMR for the patient to a record in a cloud.

19. A method, comprising:
by a smart phone,
authenticating a user based on authorization from a medical practice;
performing text recognition on image data of a label on a pharmaceutical sample;
performing a comparison operation with the image data and a pre-loaded inventory of pharmaceutical samples;
displaying matched data including a medication name, dose, lot number, and expiration date, responsive to performing the comparison operation;
receiving a user confirmation of the matched data; and
updating the inventory of pharmaceutical samples based on the user confirmation.

20. The method of claim 19, further comprising:
by the smart phone,
receiving a patient identification for a patient;
referencing stored electronic medical records to find a medical record for the patient;
determining whether the patient has an allergy associated with the matched data;
and generating an alert notification when the patient has an allergy associated with the matched data.
